(19) United States
(12) Patent Application Publication
(10) Pub. No.: US 2023/0301701 A1
(43) Pub. Date: Sep. 28, 2023

(54) ELECTROSURGICAL GENERATOR

(71) Applicant: OLYMPUS WINTER & IBE GMBH, Hamburg (DE)

(72) Inventors: Stefan DIETRICH, Potsdam (DE); Jens KRÜGER, Hamburg (DE); Fabian JANICH, Hamburg (DE); Fabian STOPP, Berlin (DE); Anne KWIK, Berlin (DE)

(73) Assignee: OLYMPUS WINTER & IBE GMBH, Hamburg (DE)

(21) Appl. No.: 18/109,659

(22) Filed: Feb. 14, 2023

Related U.S. Application Data
(60) Provisional application No. 63/324,233, filed on Mar. 28, 2022.

(51) Int. Cl. A61B 18/12; G06F 3/01

(52) U.S. Cl. CPC A61B 18/1206; G06F 3/012; G06F 3/013; G06F 3/017; G06V 40/166

(57) ABSTRACT

An electrosurgical generator includes a control unit, an electrosurgical function unit, and a user interface unit, wherein the electrosurgical function unit is configured to provide an electrosurgical therapy signal to one or more electrosurgical devices, the control unit is configured to control operation of the electrosurgical function and user interface units, and the user interface unit is configured to receive status information data from the control unit and to output that information to a user and allow input of user input data and to communicate that data to the control unit; wherein the user interface unit includes a proximity sensor configured to detect the presence of a user within a predetermined proximity of the user interface unit and to output a detection signal to the control unit, and the control unit is configured to switch the electrosurgical generator between first and second operational modes in response to the signal.

### FIELD OF THE INVENTION

The present disclosure is related to electrosurgical generators. More specifically, the disclosure is related to the use of proximity sensors for controlling electrosurgical generators.

### BACKGROUND

In modern surgery, electrosurgical instruments are used to perform or assist with a plurality of different surgical procedures. Electrosurgical instruments use electric currents, mostly high-frequency alternating currents, to create a desired effect in tissue under treatment. Depending on the desired outcome, tissue effects can include one or more of coagulation, desiccation, evaporation, and cutting. In a special variation of electrosurgery, high-frequency electrical currents are converted into ultrasonic vibrations through a sonotrode, which then are used to create a tissue effect. Electrical currents for use in electrosurgery are commonly referred to as electrosurgical therapy signals.

Electrosurgical therapy signals are usually provided by electrosurgical generators. Such electrosurgical generators are highly sophisticated medical devices comprising a control unit, an electrosurgical function unit, and a user interface unit.

The electrosurgical function unit is configured to provide electrosurgical therapy signals to one or more electrosurgical instruments. Depending on the desired tissue effect, the electrosurgical function unit may control various parameters of the electrosurgical therapy signal like voltage, current, power, waveform, frequency, pulse-pause ratio, and the like. The electrosurgical function unit may further be configured to monitor the reaction of tissue under treatment by measuring tissue impedance, tissue temperature, tissue moisture, or the like. Such measurement can be performed through dedicated sensors associated with electrosurgical instruments, and/or through indirect measurement based on electrical characteristics of the electrosurgical therapy signal.

The control unit is configured to control operation of the electrosurgical function unit. Therefore, the control unit may communicate information regarding parameters of the electrosurgical therapy signal to the electrosurgical function unit. The control unit may further communicate activation/deactivation commands to the electrosurgical function unit to activate or deactivate output of the electrosurgical therapy signal. The electrosurgical function unit may communicate status information and tissue reaction information to the control unit.

The user interface unit is configured to receive status information data from the control unit and to output that status information data to a user, and to allow input of user input data from a user and to communicate that user input data to the control unit. The user interface unit may comprise an output device like an electronic display, and one or more input devices like buttons, switches, or knobs. The user interface unit may comprise a combined input/output device like a touchscreen.

In many surgical procedures, the use of electrosurgery is part of the planned procedure. In such procedures, the electrosurgical generator can be brought into fully activated operational mode at the beginning of the procedure. In other procedures, particularly in non-invasive procedures, an electrosurgical system including an electrosurgical generator and electrosurgical instruments are provided as a backup to be available in case of complications. For example, if tissue lesions are observed during an endoscopic examination of the gastric tract, it may be necessary to apply electrosurgery to take biopsies or to fully excise such lesions. In other examples, unexpected bleedings may occur during a proceeding, and electrosurgery needs to be applied for controlling such bleeding. In such usually non-invasive procedures, it may be inefficient or otherwise undesirable to keep an electrosurgical generator in a fully activated operational mode for the whole time.

However, in case of complications as described above, the electrosurgical generator needs to be brought into a fully activated operational mode fast. In this case, a medical practitioner or assistant needs to turn to the electrosurgical generator, identify the correct input device of the user interface unit for changing the operational mode of the electrosurgical generator, and to operate that input device. This may cause a delay in activation of the electrosurgical generator, which may negatively affect the outcome of the procedure.

It is an object of the present disclosure to provide an improved electrosurgical generator.

### SUMMARY OF THE DISCLOSURE

The present disclosure provides an electrosurgical generator, comprising a control unit, an electrosurgical function unit, and a user interface unit, wherein the electrosurgical function unit is configured to provide an electrosurgical therapy signal to one or more electrosurgical devices, the control unit is configured to control operation of the electrosurgical function unit and the user interface unit, and the user interface unit is configured to receive status information data from the control unit and to output that status information to a user and allow input of user input data from a user and to communicate that user input data to the control unit; wherein the user interface comprises a proximity sensor configured to detect the presence of a user within a predetermined proximity of the user interface unit and to output a detection signal to the control unit, and the control unit is configured to switch the electrosurgical generator between a first operational mode and a second operational mode in response to the detection signal.

An electrosurgical generator according to the present disclosure may be switched between a first operational mode and a second operational by a user without the user needing to identify and operate a dedicated input device. Thereby, the activation of the electrosurgical generator in case of an unexpected situation or complication may be effected much faster than with a prior art electrosurgical generator.

The proximity sensor may include a time-of-flight (TOF) sensor. TOF sensors are designed to measure the travelling time of photons emitted by the sensor and reflected from a target in order to measure the distance between the sensor and the target. The proximity sensor may include a video camera. The proximity sensor may use focus information of an image taken by the video camera to determine a distance or distance range between a target and the video camera. The video camera may be a stereoscopic camera. The proximity sensor may include a TOF camera. A TOF camera combines the concepts of a TOF sensor and a video camera and is able to provide 3D image information wherein each pixel of an image is assigned one or more brightness values and a distance value indicating the distance between an object in the image and the TOF camera.

The proximity sensor may further include an image processor configured to receive 2D or 3D image data from the video camera or the TOF camera, apply a face detection algorithm for detecting presence of a human face in the 2D or 3D image data, and generate the detection signal if a human face is detected in the 2D or 3D image data. Various face detection algorithms are known to the skilled person, and need not to be explained in detail, here.

The image processor may further be configured to apply a head pose detection algorithm when a human face is detected in the 2D or 3D image data, and to generate an attention signal when the human face detected in the 2D or 3D image data is turned towards the user interface. Several head pose detection algorithms are known to the skilled person, and need not be explained in detail, here.

The image processor may further be configured to apply a gaze detection algorithm when a human face is detected in the 2D or 3D image data, and to generate a gaze signal indicating a viewing direction of the human face. Several gaze detection algorithms are known to the skilled person, and need not to be explained in detail, here.

The image processor may further be configured to apply a gesture detection algorithm when a human face is detected in the 2D or 3D image data, and to generate a gesture signal indicating a gesture performed by the human whose face is detected in the 2D or 3D image data. Possible gestures may include facial gestures, i.e. gestures only preformed with the face, like blinking with one or both eyes, hand gestures, or full-body gestures. Gesture detection algorithms are well known to the skilled person, and need not to be explained in detail, here. The image processor may be configured to apply a gesture detection algorithm independently from the detection of a face in the 2D or 3D image data. In this case, the image processor may detect gestures e.g. hand gestures, even if a face of the person performing the gesture is outside of a field of vision of the video camera of the TOF camera.

The control unit may be configured to switch the electrosurgical generator between two or more operational modes in response to the detection signal and one or more of the attention signal, the gaze signal, and the gesture signal. The one or more operational modes include one or more of: a standby mode, in which the control unit and the user interface unit are active, and in which the electrosurgical function unit is inactive; an active mode, in which the control unit, the user interface unit, and the electrosurgical function unit are active; a screensaver mode, in which a display of the user interface unit is deactivated or activated to display a predetermined screensaver image or image sequence; a status display mode, in which the user interface is controlled to display status information of the electrosurgical generator on the display; and a user input mode, in which the user interface is controlled to display one or more interactive user input elements on the display, and to receive user input through the one or more user input elements.

Some examples of the present disclosure are described in the following at hand of illustrative drawings. The examples described are provided for better understanding, and are not supposed to be exhaustive, or to limit the scope of the appended claims in any way.

### BRIEF DESCRIPTION OF THE DRAWINGS

The drawings show:

FIG. 1: An electrosurgical system,

FIG. 2: The electrosurgical generator of the electrosurgical system of FIG. 1,

FIG. 3: A schematic design of a proximity sensor using a video camera,

FIG. 4: A schematic design of a further proximity sensor,

FIG. 5: An image processing algorithm.

### DETAILED DESCRIPTION

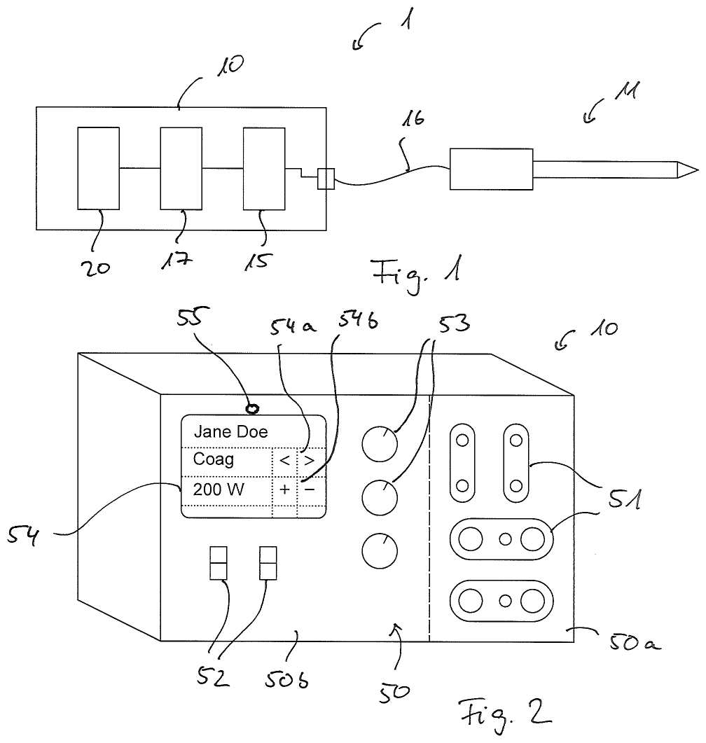

FIG. 1 shows an electrosurgical system 1 with an electrosurgical generator 10 and an electrosurgical instrument 11. The electrosurgical generator 10 comprises an electrosurgical function unit 15, which is configured to provide one or more electrosurgical therapy signals to the electrosurgical instrument 11. The electrosurgical instrument may be connected to the electrosurgical generator 10 and the electrosurgical function unit 15 through a cable 16. The electrosurgical generator 10 further comprises a control unit 17 and a user interface unit 20.

The electrosurgical function unit 15 is configured to provide electrosurgical therapy signals to the electrosurgical instrument 11. Depending on the desired tissue effect, the electrosurgical function unit 15 may control various parameters of the electrosurgical therapy signal like voltage, current, power, waveform, frequency, pulse-pause ratio, and the like. The electrosurgical function unit 15 may further be configured to monitor the reaction of tissue under treatment by measuring tissue impedance, tissue temperature, tissue moisture, or the like. Such measurement may be performed through dedicated sensors associated with the electrosurgical instrument 11, and/or through indirect measurement based on electrical characteristics of the electrosurgical therapy signal.

The control unit 17 is configured to control operation of the electrosurgical function unit 15. Therefore, the control unit 17 may communicate information regarding parameters of the electrosurgical therapy signal to the electrosurgical function unit 15. The control unit 17 may further communicate activation/deactivation commands to the electrosurgical function unit 15 to activate or deactivate output of the electrosurgical therapy signal. The electrosurgical function unit 15 may communicate status information and tissue reaction information to the control unit 17.

The control unit 17 may include a processor, memory, and associated hardware known from standard computer technology. The control unit may include program code information stored on the memory for causing the processor to perform various activities of the control unit 17 when executed by the processor. The program code information may include a standard operating system like Windows, MAC-OS, android, Linux, or the like, and/or a proprietary operating system provided by the manufacturer of the electrosurgical generator 10. Such standard computer hardware and operating systems are known to a user and need not be described in detail, here.

The user interface unit 20 is configured to receive status information data from the control unit 17 and to output that status information data to a user, and to allow input of user input data from a user and to communicate that user input data to the control unit 17. The user interface unit 20 may comprise an output device like an electronic display, and one or more input devices like buttons, switches, or knobs. The user interface unit 20 may comprise a combined input/output device like a touchscreen. The user interface unit 20 may be integrated into a housing of the electrosurgical generator 10. Some or all components of the user interface unit may be located outside of the housing of the electrosurgical generator 10. Such components may include one or more foot switches (not shown). The user interface unit 20 may comprise data processing hardware separate from the control unit 17, like a processor, memory, and the like. The user interface unit 20 may share some or all data processing hardware with the control unit 17.

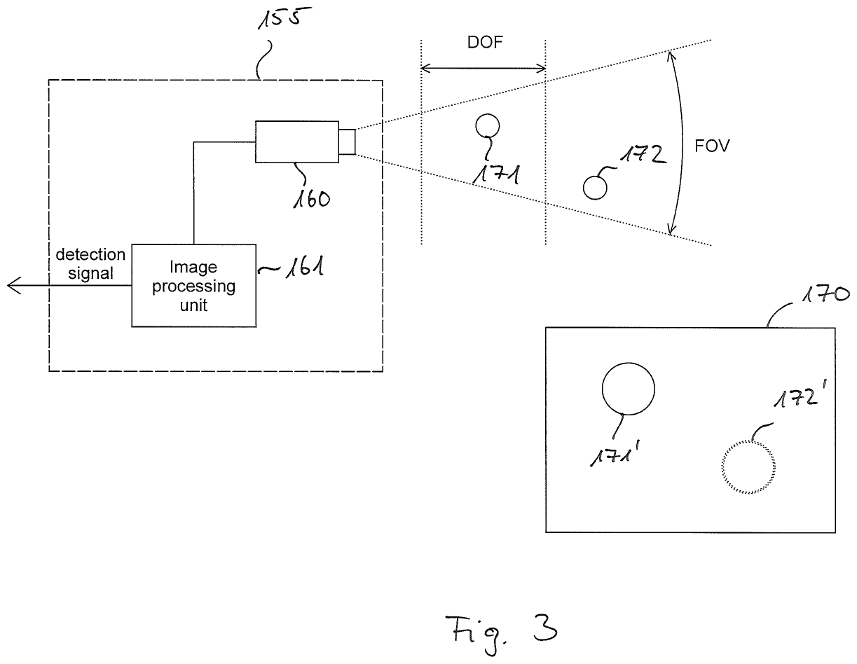

FIG. 2 shows a simplified isometric view of the electrosurgical generator 10. A front panel 50 of the electrosurgical generator 10 includes a connection section 50a and a user interface section 50b.

In the connection section 50a, a plurality of connecting elements 51 are provided, which allow connection of various electrosurgical instruments. The connection section 50a is associated with the electrosurgical function unit 15 of the electrosurgical generator 10.

In the user interface section 50b, a plurality of switches 52 and knobs 53 are provided, which allow input of user input data through operation of the switches 52 and/or knobs 53. A display element 54 is provided for outputting of status data. In the shown example, the status data includes a patient name, a selected tissue effect, and a selected output power of an electrosurgical therapy signal. The selection of status data items shown in FIG. 2 is just an example. Some of the status data items, like the patient name, may be omitted. Other status data elements may be displayed on the display element 54. The display element 54 may be a touchscreen, allowing input of further user input data though activation of interactive display elements like “left”/“right” buttons 54a for selecting different tissue effects, or “+”/“−” buttons 54b for increasing or decreasing the selected output power The user interface section 50b further includes a proximity sensor 55, which will be described in more detail below. The user interface section 50b is associated with the user interface unit 20 of the electrosurgical generator 10.

The proximity sensor 55 is configured to detect the presence of a user within a predetermined proximity of the user interface unit 20, or the user interface section 50b associated therewith. The predetermined proximity may be a range of 1 m, 2 m, 3 m, or any other appropriate proximity range. The proximity sensor 55 is configured to output a detection signal to the control unit 17. The detection signal may be a binary signal having a value of “1” if a user has been detected in the predetermined proximity range, and a value of “0” when no user has been detected. The detection signal may include a numeric value indicating the distance between a detected user and the user interface unit 20.

The control unit 17 is configured to switch the electrosurgical generator 10 between a first operational mode and a second operational mode in response to the detection signal.

The proximity sensor 55 may be an ultrasonic proximity sensor. An ultrasonic proximity sensor may emit ultrasonic waves, receive ultrasonic waves reflected from an object within the propagation path of the ultrasonic waves, and determine the distance between the proximity sensor and the object based on a travelling time of the ultrasonic waves.

The proximity sensor 55 may be an electromagnetic proximity sensor. Electromagnetic proximity sensors may include laser sensors, radar sensors, lidar sensors, or the like. The proximity sensor 55 may be an optical time-of-flight (TOF) sensor.

TOF sensors typically include a light emitter, a light detector, and a timer. The light emitter emits a modulated stream of photons, e.g. a pulsed laser beam. The light detector detects light reflected from an object within the propagation path of the laser beam, and the timer is used to determine how much time elapsed between emission and detection of the light. The distance between the TOF sensor and the object can then easily be calculated from the elapsed time and the known speed of light.

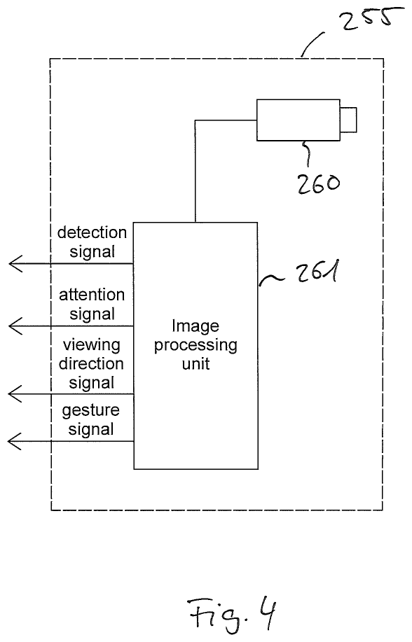

The proximity sensor 55 may include a video camera. The schematic design of a proximity sensor 155 including a video camera is shown in FIG. 3. The proximity sensor 155 includes a video camera 160 and an image processing unit 161. The video camera 160 is configured to acquire an image 170 of the vicinity of the electrosurgical generator 10 within a certain field of view (FOV). The video camera 160 comprises an objective lens system (not shown) and an electronic image converter (not shown), as commonly known in the art. The objective lens system may be configured to provide a certain depth of field (DOF), so that objects within the predetermined proximity of the user interface unit 20, or the user interface section 50b associated therewith, are depicted sharp in the acquired image 170, while objects outside of the predetermined proximity are blurred. In FIG. 3, a first object 171 is situated within the DOF, so that a representation 171′ of object 171 in the image 170 is sharp. A second object 172 is situated outside of the DOF, so that a representation 172′ of the object 172 in the image 170 is blurred.

The image processing unit 161 is configured to analyse the image 170 and to identify objects within the DOF. Therefore, the image processing unit may apply known image analysis algorithms like an object identification algorithm for identifying representations of discrete objects (171′, 172′) in the image 170, and an image sharpness algorithm for determining whether the representations of the objects (171′, 172′) in the image 170 are sharp or blurred.

The image processing unit 161 may further be configured to generate a detection signal when at least one sharp representation of an object has been found in the image 170.

The video camera 160 may be a stereoscopic video camera. A stereoscopic video camera usually acquires two images of a scene from slightly different viewing directions, allowing determination of the distance of an object from the video camera. In this case, the image processing unit 161 may be configured to generate a detection signal comprising a numeric value indicating the distance between a detected object and the video camera 160.

The video camera 160 may be a TOF camera. A TOF camera combines the principles of a video camera and a TOF sensor, so that the TOF camera is able to acquire a 3D image. In a 3D image acquired by a TOF camera, each pixel of the image comprises brightness information and distance information of the object depicted in each respective pixel. Again, in this case the image processing unit 161 may be configured to generate a detection signal comprising a numeric value indicating the distance between a detected object and the video camera 160.

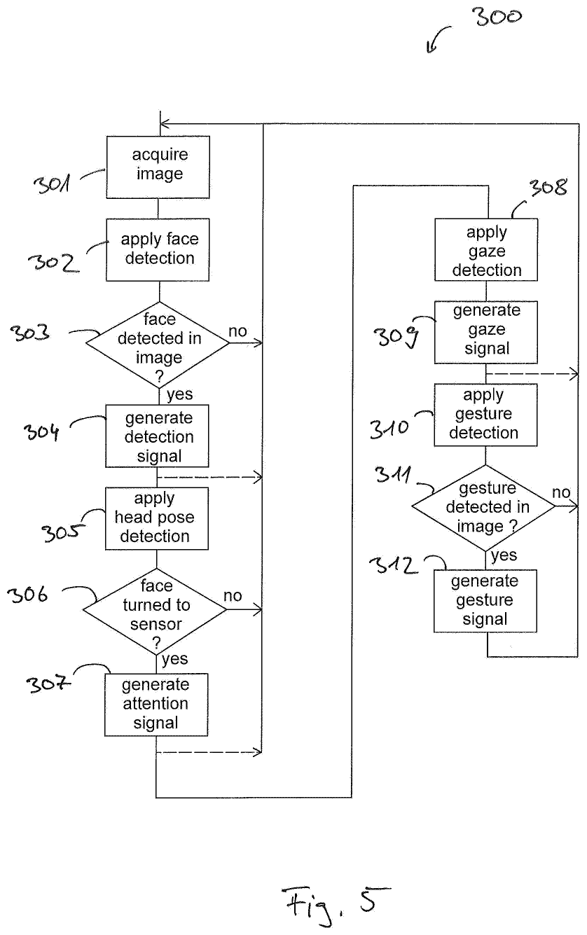

FIG. 4 shows a further possible example of a proximity sensor 255. The proximity sensor 155 comprises a video camera 260, which may again be a 2D video camera, a stereoscopic video camera, or a TOF camera, and an image processing unit 261. The image processing unit 261 is configured to analyse an image acquired by the video camera 260, and to generate a number of signals derived from the image. Therefore, the image processing unit may perform an image processing algorithm 300 as shown in FIG. 5. The signals to be generated by the image processing unit may include a detection signal, an attention signal, a gaze signal, and a gesture signal.

The image processing algorithm 300 starts with the acquisition of an image in step 301. The image acquired may be a 2D image, a stereoscopic 3D image, or a TOF 3D image.

In step 302, a face detection algorithm is applied to detect human faces in the acquired image. The face detecting algorithm may scan the acquired image for predetermined image patterns typical for human faces. Such face detection algorithms are well known in the art. In case of a 2D image, the face detection algorithm may be configured to identify faces only when they are depicted sharply, i.e. if the face is within the DOF of the video camera. In case of a 3D image, the face detection algorithm may be designed to apply a distance filter, so that only faces within a predetermined distance range from the proximity sensor 255 are detected.

In step 303, it is checked whether a face has been detected within the acquired image. If no face has been detected, the algorithm loops to acquire a new image in step 301. If a face has been detected, a detection signal is generated in step 304. Thus, it is avoided that detection signals are issued if other objects are accidentally brought into proximity with the electrosurgical generator 100, which do not indicate an intended user interaction.

After step 304, the algorithm may loop to step 301 to acquire a new image. Optionally, the algorithm may continue to apply a head pose detection algorithm in step 305. The head pose detection algorithm may be configured to determine whether the face detected in step 302 is turned towards the proximity sensor 255. Head pose detection algorithms are well known in the art. The head pose detection algorithm may be configured to detect the position of landmarks in a detected face image, like eyes, nose, ears, and the like, and to determine the pose of the head accordingly.

In step 306, it is checked whether the face detected in step 302 is turned towards the proximity sensor 255. If the face is turned towards the proximity sensor 255, an attention signal is generated in step 307. If the face is not turned towards the proximity sensor 255, the algorithm loops to acquire a new image in step 301.

In a further optional extension of the disclosed algorithm, a gaze detection algorithm may be applied in step 308, and a gaze signal may be generated in step 309. Gaze detection algorithms are well known in the art. The gaze detection algorithm may be configured to analyse the position of an iris within an eye in the image of the face. The gaze signal may use spherical coordinates to indicate azimuth and elevation angles of a detected viewing direction of the face detected in step 302. The gaze signal may use coordinates of a virtual rectangular grid in a plane of the proximity sensor 255, the plane being rectangular to the optical axis of the video camera 260.

In an even further optional extension of the disclosed algorithm, a gesture detection algorithm may be applied in step 310. Gesture detection algorithms are known in the art. A gesture detection algorithm may be configured to detect the presence of facial gestures, hand gestures, or full body gestures, in the image acquired in step 302. In step 311 it may be checked whether or not a gesture has been detected. If a gesture has been detected, a gesture signal is generated in step 312. The gesture signal may comprise a numerical code identifying the detected gesture. If no gesture has been detected, or after generation of the gesture signal, the algorithm loops to acquire a new image in step 301. The gesture detection algorithm may be applied independently from the detection of a face in step 303.

The disclosed algorithm is performed by a processor using instructions stored in a memory associated with the processor. The processor and memory may be part of an integrated proximity sensor device. The processor and memory may be part of the user interface unit 20 or the control unit 17.

The control unit 17 is configured to switch the electrosurgical generator 10 between two or more operational modes in response to signals generated by the proximity sensor 55, 155, 255. Such operational modes are described below.

Standby mode: In a standby mode of the electrosurgical generator 10, the control unit 17 and the user interface unit 20 may be active, while the electrosurgical function unit 15 may be inactive. More specifically, the control unit 17 may have completed any necessary start-up routines which may be necessary after powering on the electrosurgical generator. The user interface unit may be in an active mode idling for input of user input date. The proximity sensor 55, 155, 255 of the user interface unit 20 may be active and, for example may execute the algorithm 300 in a loop. The electrosurgical function unit 15 may be inactive, so that the total power consumption of the electrosurgical generator 10 is reduced. With the electrosurgical function unit 15 being inactive, the electrosurgical generator 10 is also less susceptible to causing or suffering electromagnetic disturbances, compared to the electrosurgical function unit 15 being active.

Active mode: In an active mode, the electrosurgical function unit 15 may also be active. This may include the electrosurgical function unit 15 being active, but not actually providing any electrosurgical therapy signals.

Screensaver mode: In a screensaver mode, the display element 54 may be switched off to reduce power consumption of the user interface unit, and/or to reduce thermal stress of the display element. In a screensaver mode, the display element may be controlled to display a predetermined screensaver pattern or pattern sequence to avoid “burning in” of a certain image on the display element 54.

Status display mode: In a status display mode, the display element 54 may be controlled to display status information like a currently selected tissue effect or output power, a current duration of application of an electrosurgical therapy signal, an accumulated applied energy, a tissue status, or the like. In a status display mode, the user interface unit 20 may offer no or limited possibilities for input of user input data. More specifically, some or all of the switches 52 and knobs 53 may be deactivated in a status display mode.

User input mode: In a user input mode, the user interface unit 20 may be configured to allow input of user input data. The display unit 54 may be configured to display current values of various parameters, and may further display interactive control elements like virtual buttons 54a, 54b which can be operated by a user by touching the display element, or by using an input device like a mouse, a touchpad, or a trackball. In a user input mode, the switches 52 and knobs 53 may be activated to allow input of user input data by a user.

The control unit 17 may be configured to switch the electrosurgical generator 10 from a standby mode into an active mode in response to a detection signal generated by the proximity sensor 55, 155, 255. More specifically, the control unit 17 may be configured to switch the electrosurgical generator 10 from a standby mode into a user input mode in response to the detection signal.

The control unit 17 may be configured to switch the electrosurgical generator 10 from a standby mode into an active mode only when the proximity sensor 155, 255 generates both a detection signal and an attention signal. This helps to prevent unnecessary switching of the electrosurgical generator 10 in cases where a person walks by the proximity sensor 255, but does not look at the electrosurgical generator 10.

The control unit 17 may be configured to switch the electrosurgical generator 10 from a status display mode or from a user input mode into a screen saver mode if the proximity sensor 155, 255 stops to generate a detection signal and/or an attention signal.

The control unit 17 may be configured to switch the electrosurgical generator 10 from a screen saver mode into a user input mode or a status display mode if the proximity sensor 255 starts generating a detection signal and/or an attention signal.

The control unit 17 may be configured to switch the electrosurgical generator 10 into a status display mode when the proximity sensor 155, 255 generates a gaze signal indicating that a user is looking towards the connecting elements 51. The control unit 17 may be configured to switch the electrosurgical generator 10 into a user input mode when the proximity sensor 155, 255 generates a gaze signal indicating that a user is looking towards the switches 52 or knobs 53.

The control unit 17 may be configured to change one or more user input parameters when the electrosurgical generator 10 is in a user input mode, and when the proximity sensor 255 generates one of a plurality of predetermined gesture signals.

1. An electrosurgical generator, comprising a control unit, an electrosurgical function unit, and a user interface unit, wherein
the electrosurgical function unit is configured to provide an electrosurgical therapy signal to one or more electrosurgical devices,
the control unit is configured to control operation of the electrosurgical function unit and the user interface unit, and
the user interface unit is configured to
receive status information data from the control unit and to output that status information to a user and
allow input of user input data from a user and to communicate that user input data to the control unit;
wherein
the user interface unit comprises a proximity sensor configured to detect the presence of a user within a predetermined proximity of the user interface unit and to output a detection signal to the control unit, and
the control unit is configured to switch the electrosurgical generator between a first operational mode and a second operational mode in response to the detection signal.

2. The electrosurgical generator of claim 1, wherein the proximity sensor includes a time-of-flight (TOF) sensor.

3. The electrosurgical generator of claim 1, wherein the proximity sensor includes a video camera.

4. The electrosurgical generator of claim 2, wherein the proximity sensor includes a TOF camera.

5. The electrosurgical generator of claim 3, wherein the proximity sensor further includes an image processor configured to
receive 2D or 3D image data from the video camera or the TOF camera,
apply a face detection algorithm for detecting presence of a human face in the 2D or 3D image data, and
generate the detection signal if a human face is detected in the 2D or 3D image data.

6. The electrosurgical generator of claim 5, wherein the image processor is further configured to apply a head pose detection algorithm when a human face is detected in the 2D or 3D image data, and to generate an attention signal when the human face detected in the 2D or 3D image data is turned towards the user interface.

7. The electrosurgical generator of claim 5, wherein the image processor is further configured to apply a gaze detection algorithm when a human face is detected in the 2D or 3D image data, and to generate a gaze signal indicating a viewing direction of the human face.

8. The electrosurgical generator of claim 5, wherein the image processor is further configured to apply a gesture detection algorithm when a human face is detected in the 2D or 3D image data, and to generate a gesture signal indicating a gesture performed by the human whose face is detected in the 2D or 3D image data.

9. The electrosurgical generator of claim 5, wherein the control unit is configured to switch the electrosurgical generator between two or more operational modes in response to the detection signal and one or more of the direction signal, the gaze signal, and the gesture signal.

10. The electrosurgical generator of claim 1, wherein the one or more operational modes include one or more of:
a standby mode, in which the control unit and the user interface unit are active, and in which the electrosurgical function unit is inactive;
an active mode, in which the control unit, the user interface unit, and the electrosurgical function unit are active;
a screensaver mode, in which a display element of the user interface unit is deactivated or activated to display a predetermined screensaver image or image sequence;
a status display mode, in which the user interface is controlled to display status information of the electrosurgical generator on the display element; and
a user input mode, in which the user interface is controlled to display one or more interactive user input elements on the display element, and to receive user input through the one or more user input elements.
